(19) United States
(12) Patent Application Publication
(10) Pub. No.: US 2010/0267058 A1
(43) Pub. Date: Oct. 21, 2010

(54) CIRCULATING RET RECEPTOR

(75) Inventors: Peter Hamer, Reading, MA (US); Stephen P. Bradley, Brighton, MA (US); Walter Carney, North Andover, MA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Healthcare Diagnostics Inc., Tarrytown, NY (US)

(21) Appl. No.: 12/808,708

(22) PCT Filed: Dec. 17, 2008
(86) PCT No.: PCT/US08/87142

§ 371 Date: Jun. 17, 2010

Related U.S. Application Data
(60) Provisional application No. 61/015,700, filed on Dec. 21, 2007.

(51) Int. Cl. G01N 33/574; C07K 16/30

(52) U.S. Cl. 435/7.23; 530/389.7

(57) ABSTRACT

Provided are compositions, methods, and kits relating to the detection of a circulating or soluble form of the KET receptor tyrosine kinase.

### FIELD OF THE INVENTION

Provided are compositions, methods, and kits relating to the detection of a circulating or soluble form of the RET receptor tyrosine kinase.

### BACKGROUND OF THE INVENTION

The RET receptor gene on chromosome 10q11.2 encodes a tyrosine kinase transmembrane receptor. The RET receptor consists of three functional domains: a large intracellular tyrosine kinase domain, a transmembrane region, and an extracellular domain having four cadherin-like repeats that are implicated in ligand binding and a cysteine rich region. Drosten M & Pützer BM, Nat Clin Prod Oncol. 3(10):564-74 (2006). Review. The RET receptor gene is expressed in tissues of neural crest origin, including the sympathetic ganglia, adrenal medulla, thyroid C cells, and excretory system of the developing kidney. Cerchia L et al., Biochem J. 2003 Jun. 15; 372(Pt 3):897-903.

Mutations in the RET receptor extracellular region have been implicated in multiple endocrine neoplasia types 2A and 2B (MEN2A and MLN2B) syndrome, familial medullary thyroid carcinoma and Hirschsprung's disease (HSCR), a congenital disorder of the colon. Id. See also Kodama Y et al., Cancer Sci 2005 March, 96(3):143-8. Oncogenic RET receptor induces transforming activity and promotes cell invasiveness. Id. (citations omitted). Mutations in the cysteine-rich domain of specific cysteine residues convert RET receptor into a dominant transforming gene and induce constitutive activation of its intrinsic tyrosine kinase activity, which leads to congenital and sporadic cancers in neuroendocrine organs. Takahashi M, Cytokine Growth Factor Rev. 12, 361-373 (2001); Santoro M, et al., Science 267, 381-383 (1995); Hansford J R & Mulligan L M, J. Med. Genet. 37, 817-827 (2000). Additionally, deletions and point mutations distributed along the entire RET receptor gene have been described in sporadic and familial cases of HSCR. Cerchia L et al., Biochem J. 2003 Jun. 15; 372(Pt 3) 897-903 (citations omitted). Both germline and somatic mutations in RET receptor are known to be causative for the development of medullary thyroid carcinoma (MTC), and strong evidence supports the notion that inhibition of RET receptor oncogene function represents an option for the treatment of MTC. Drosten M& Pützer BM, Nat Clin Pract Oncol. 300):564-74 (2006). Review. MTC accounts for 5-10% of all thyroid carcinomas, and the 10-year survival rate for this cancer has been estimated at about 60-70%. Id. (citations omitted). Treatment is most successful when the disease is localized strictly to the thyroid gland, which makes early detection critical for positive treatment results.

RET receptor (RETr) is considered to be a prime target for various treatment strategies. It has been proposed that the development of a monoclonal antibody to the ectodomain RET receptor could aid treatment of RETr-associated cancer. Kodama Y, et al., Cancer Sci. 2005 March; 96(3):143-8. A small molecule tyrosine kinase inhibitor also represents an important option for anticancer treatment. See Santoro M, et al., Endocrinology. 2004 December; 145(12):5448-51. Review.

The RET receptor has therefore emerged as promising diagnostic benchmark and target for preclinical and clinical therapeutic applications with respect to RET-associated cancer and other conditions. Assessments of RET receptor expression in patient samples could have broad applicability for any or all of diagnosis, monitoring, and treatment of cancer and cancer-related pathologies.

### SUMMARY OF THE INVENTION

Provided are methods comprising detecting a soluble form of RET receptor in a biological fluid of a subject.

Also provided are methods for detecting or aiding the detection of a disease state in a patient comprising determining the quantity of soluble RET receptor in a biological fluid of the patient, wherein a quantity of soluble RET receptor that is at variance with a reference normal value indicates the presence of the disease state in the patient.

There are also disclosed methods for monitoring a patient undergoing a therapy regimen comprising determining a plurality of values corresponding to the quantity of soluble RET receptor in a biological fluid of the patient over time and determining whether a change hi said values over time has occurred.

Also provided are methods for monitoring a patient in which a disease state is known to be present comprising determining the quantity of soluble RET receptor in a biological fluid of said patient, thereby obtaining a sample value, and comparing said sample value with a reference value.

The present invention is also directed to purified antibodies that specifically bind to a soluble form of RET receptor, as well as kits for detecting a soluble form of RET receptor in a biological fluid comprising the purified antibody for use as a detector reagent; and, instructions for use comprising a standard curve for interpolating the quantity of the soluble form of RET receptor in the biological fluid.

### BRIEF DESCRIPTION OF THE DRAWINGS

FIG. 1 depicts measurements of soluble RET receptor in normal serum and plasma samples.

FIG. 2 depicts measurements of soluble RET receptor in serum and plasma samples of subjects in which cancer is known to be present.

### DETAILED DESCRIPTION OF ILLUSTRATIVE EMBODIMENTS

While the RET receptor has been characterized in numerous studies, circulating forms of RET are heretofore unknown in the literature. The present invention pertains to the novel identification of a circulating form of the REF receptor tyrosine kinase, assays for the detection and quantification of circulating RET receptor, and diagnostic, monitoring, and treatment methodologies that are effected through such detection, quantification, or both. As used herein, the terms “soluble” and “circulating” are interchangeable and applied in connection with the newly discovered non-membrane-bound form of RET receptor.

The role of mutated membrane-bound RET receptor in the generation of metastatic phenotype has been confirmed through numerous studies, and as a result the RET receptor has been proposed as a promising target for diagnosis and therapeutic intervention with respect to a variety of carcinomas and metastatic mechanisms, such as cell growth, cell migration, and angiogenesis. Pursuant to the present invention, a naturally occurring form of soluble RET receptor has been discovered for the first time. The soluble version of RET receptor represents an independent prognostic and evaluative marker for carcinomas and the mechanisms of metastasis. The utility of biomarkers to manage patient care has been firmly established, for example, in such systems as Her2/neu and Herceptin, EGFr, and IRESSA. The present discovery of naturally-occurring soluble RET receptor therefore tenders new opportunities for diagnostic and therapeutic applications relating to numerous cancers and cancer-related phenotypes, among other conditions.

In the present disclosure the singular forms “a,” “an,” and “the” include the plural reference, and reference to a particular numerical value includes at least that particular value, unless the context clearly indicates otherwise. When values are expressed as approximations, by use of the antecedent “about,” it will be understood that the particular value forms another embodiment. Where present, all ranges are inclusive and combinable.

Provided are methods comprising detecting a soluble form of RET receptor in a biological fluid of a subject. The phrase “soluble form of RET receptor” refers to a complete molecule of RET receptor tyrosine kinase that exists in circulating form, or a circulating fragment of the RET receptor tyrosine kinase, such as the extracellular domain portion of the RET receptor tyrosine kinase, or a fragment of the extracellular domain portion. In preferred embodiments, “soluble form of RET receptor” refers to at least the portion of the RET receptor that includes one or more of the epitopes recognized by the commercially available anti-human RET receptor polyclonal antibody (R&D Systems, Minneapolis, Minn.; Cat. No. AF1485). “Soluble form of RET receptor” may also mean a molecule of RET receptor tyrosine kinase, whether complete or a fragment, having one or more of any of the mutations observed with respect to the membrane-bound form of the RET receptor. See, e.g., Santoro M, et al., Endocrinology. 2004 December; 145(12) 5448-51 Review; Drosten & Pützer BM, Nat Clin Pract Oncol 3(10):564-74 (2006). Review; Santoro et al., Ann. N.Y. Acad. Sci. 963:116-121 (2002). In accordance with the present invention, soluble RET receptor may be detected in an acellular biological fluid, or in a biological fluid containing cells or cell fragments. Exemplary acellular biological fluids include, for example, acellular serum or plasma, sputum, cell-free cerebrospinal fluid, acellular saliva, sweat, tears, and urine. Whole blood, cellular fractions thereof, cell-containing saliva, tumor lysates, cerebrospinal fluid, or liquefied tissue samples are exemplary biological fluids that contain cells. Biological fluids may be obtained by conventional methods, such as biopsy to obtain tissue samples, venipuncture to obtain blood, and fractionating whole blood in order to obtain serum or plasma.

Detection of a soluble form of RET receptor can be carried out using one or more assays that are known to effect the identification of proteins in a biological sample. Immunohistochemical techniques are preferred. For example, in one embodiment, a reagent comprising a monoclonal antibody that is specific to the extracellular domain of the soluble form of RET receptor is incubated with the biological sample, followed by incubation with a secondary antibody that binds to the monoclonal antibody and that is conjugated to a detection moiety such as a fluorophore. Detection of the fluorophore is indicative of the presence of the soluble form of RET receptor. Various acrylamide gel protein detection methods may also be used. Mass spectrometry is another widely-used protein detection method that may be used in accordance with the present invention. At least some forms of soluble RET receptor may be functional, and kinase assays (i.e., measuring the phosphorylation of known RET receptor substrates (see, e.g., Santoro M, et al., Endocrinology. 2004 December; 145(12):5448-51. Review) and/or binding assays for known RET receptor substrates can constitute suitable means for detecting the presence of functional forms of soluble RET receptor. Other assays may involve automated platforms, for example, that use magnetic particles to increase assay timing. Such assays are routine and readily designed and executed by those skilled in the art.

Further to the detection of the soluble form of RET receptor, the present methods may additionally comprise determining the quantity of circulating RET receptor, such as to obtain a “sample value” for the biological fluid sample. As in the case of detection of a protein in a biological sample, those skilled in the art will readily appreciate that numerous techniques are available for the quantification of a protein in such sample. Immunohistochemical methods, mass spectrometry, flow cytometry, kinase assays, binding assays, and gel electrophoresis, among other techniques, may suitably be used. Pursuant to the present invention, a novel enzyme-linked immunosorbent assay (“ELISA”) has been developed specifically for determining the quantity of circulating RET receptor, as described in Example 1, infra.

It has herein been discovered that specific trends in RET receptor expression as indicated by the level of circulating RET receptor protein exist for normal versus cancer subjects, among different, commonly-occurring types of cancer, and with respect to different stages of the same cancer type. These results indicate that naturally-occurring soluble RET receptor, whether complete RET receptor proteins, represents an important new metric or biomarker for the potential, presence, and/or progression, of certain disease states, most notably cancer-related disease states.

The quantity of soluble RET receptor in a biological fluid of a subject can also reveal information relevant to the suitability of RET receptor-targeted therapies. Previous studies have revealed that the pretreatment level of soluble biomarker can indicate whether therapies that target the cellular form of such biomarker are likely to be efficacious. For example, it is known that if pretreatment levels of circulating Her2/neu receptor, a receptor tyrosine kinase and member of the ErbB protein family, decrease within one to two weeks following Herceptin administration, then therapy that targets cellular Her2/neu receptor is not expected to produce positive results. See Köstler W J, et al., Clin Cancer Res. 2004 Mar. 1; 10(5): 1618-24; Lipton A, et al., Clin Cancer Res. 2004 Mar. 1; 10(5):1559-60; Lipton A, et al., J Clin Oncol. 2003 May 15; 21(10):1967-72; Carney W P, et al., Clin Chem. 2003 October; 49(10) 1579-98. Review; Carney, WP, Personalized Medicine 2005; 2(4):317-324. The present invention is also directed to assessing the suitability of a therapy, such as a therapy that targets cellular RET receptor, based on the quantity of soluble RET receptor in a biological fluid of a subject, wherein the subject has not yet undergone such therapy.

Pretreatment levels of biomarker can also correlate to patient prognosis with respect to a disease state, such as predisposition for a particular rate of progression, certain outcome, or likelihood of recurrence following treatment. Overexpression of the Her2/neu receptor in breast cancer is associated with increased disease recurrence and worse overall prognosis. Lipton A, et al., Clin Cancer Res. 2004 Mar. 1; 10(5) 1559-60; Lipton A, et al., J Clin Oncol, 2003 May 15; 21(101967-72; Carney W P, et al., Clin Chem. 2003 October; 49(10):1579-98. Review; Carney, WP, Personalized Medicine 2005; 2(4):317-324. The present invention may involve measuring the pretreatment quantity of soluble RET receptor in a biological fluid of a subject in order to correlate such quantity to the prognosis of the subject with respect to a disease state. The prognosis of the subject may relate to the expected rate of progression of the disease state, outcome (e.g., degree of likelihood of survival), probability of recurrence following remission or treatment, or other prospective disease state parameter.

The “disease state” corresponds to any pathological condition concerning which soluble RET receptor provides an indicator of presence, state of development, or both. The disease state may be a cancer or a cancer-related condition. Exemplary disease states include medullary thyroid carcinoma, papillary thyroid carcinoma, kidney cancer, breast cancer, ovarian cancer, bladder cancer, prostate cancer, colon cancer, and lung cancer, as well as pathogenic cell growth migration, or angiogenesis. See also Santoro M, et al., Endocrinology. 2004 December; 145(12); 5448-51, Review (discussing various conditions associated with the RET gene). The disease state may be a condition that is in any stage of development or progression, e.g., an early-stage condition that has not yet symptomatically manifested itself, a partially-developed condition, a fully developed, symptomatic pathology, a pathology that is in remission, or a disease that has undergone or is undergoing any form of treatment. A condition that is in any of the traditional existential states (i.e., prodromal, incubation, icteric, or convalescence) is a “disease state” for purposes of the present invention.

It has herein been discovered that the mean value of soluble RET receptor in serum of subjects in which colon cancer, renal cell carcinoma, late stage lung cancer, late stage prostate cancer, late stage breast cancer, and bladder cancer are known to be present is less than the mean value of soluble RET receptor in serum of subjects in which these disease states are known to be absent, data for which is presented in Example 2, infra. It has also been discovered that the mean value of soluble RET receptor in plasma of subjects in which early stage breast cancer is known to be present is greater than the mean value of soluble RET receptor in plasma of female subjects in which these disease states are known to be absent. See Example 2. In accordance with the present invention, the “reference value” may correspond to any of a number of different parameters. For example, the reference value may correspond to the maximum quantity of soluble RET receptor that is necessary to indicate the presence of a disease state, i.e., wherein a sample value that is below such reference value is indicative of the presence of a disease state. The reference value may be a single numerical value expressed in terms of, e.g., concentration (mass percentage, mass volume percentage, mass volume ratio, “parts-per” notation, molarity, and the like), with or without a standard deviation, or the reference value may comprise a range of numerical values expressed in terms of, for example, a range of concentrations, with or without standard deviations. The reference value may comprise a single data point, or may comprise an average or mean of multiple data points for the same parameter. For example, the reference value may be the quantity of soluble RET receptor that was measured in a single biological fluid sample from a patient known to have breast cancer, or the reference value may be a calculated average of values obtained from a plurality (e.g., two, five, ten, twenty, one-hundred, etc.) of biological fluid samples, each from a patient known to have breast cancer. In other embodiments, the reference value may correspond to the minimum quantity of soluble RET receptor that is necessary to indicate the presence of a disease state. In such embodiments, the “reference value” is otherwise defined as described above. Because it may be difficult, if not medically inappropriate to attempt to define a single value corresponding to a quantity of soluble RET receptor that represents a “tipping point” above or below which (depending on the disease state) the disease state can definitively be said to be present, a reference value that corresponds to a range of values is preferred.

The “reference value” may also relate to the quantity of soluble RET receptor in a biological fluid of the same subject from which the sample value is derived, but at a different point in time. For example, the reference value may correspond to the quantity of soluble RET receptor in a biological fluid of a patient at time t=1, whereas the sample value corresponds to the quantity of soluble RET receptor in an aliquot biological fluid in which a measurement was performed at time t=2, where t=2 represents a later point in time than t=1. The amount of time between times t=1 and t=2 may be one or more hours, days, weeks, months, or years. Additionally, for example, time t=1 may represent a timepoint at which the subject was not undergoing a therapy, whereas tune t=2 may represent a timepoint at which a therapy regimen has been commenced.

The comparison between the sample value and the reference value in accordance with the present invention can yield important information regarding the absence or presence of one or more specific disease states at a given time or over a period of time, the developmental status of one or more disease states, the prospective appropriateness of a given therapy regimen, the efficacy of an ongoing therapy regimen, and other matters of importance with regard to the treatment and/or monitoring of a subject. It has been surprisingly discovered that a downward trend in levels of circulating RET receptor occurs as cancer stage increased in the cases of lung cancer, prostate cancer, and breast cancer. See Example 2, infra. On the other hand, it has been discovered that the levels of circulating RET receptor increase as cancer stage increases in the case of colon cancer. See id.

A reference value to which the sample value is compared is preferably a “best matched” value, e.g. a sample value that was derived from serum should be compared with a reference value that was derived from serum, a sample value that was derived from plasma should be compared with a reference value that was derived from plasma, a sample value that was derived from, a male subject should be compared with a reference value that was derived from a male subject, a sample value that was derived from a female subject should be compared with a reference value that was derived from a female subject, a sample value that was derived from a subject having a disease state at stage 1 should be compared with a reference value that was derived from a subject having a disease state at stage 1, and the like, depending on the type of comparison being performed.

The instant methods may further comprise selecting a therapy regimen based on the comparison of the sample value and the reference value. The selection of a therapy regimen can embrace initiating a new therapy, altering an ongoing therapy, monitoring of a subject that is or is not undergoing therapeutic intervention, or taking no further action than had been undertaken prior to the comparison between the sample value and the reference value. For example, if the comparison between the sample value and the reference value reveals that the quantity of soluble RET receptor approaches the lower end of a range of values that would be expected for a subject in which a disease state is not present, the selection of a therapy regimen may comprise initiating a protocol whereby the subject is monitored every three months, e.g., by quantitating the amount of soluble RET receptor in a biological fluid of the subject once every three months and comparing the measured sample value to a reference value following each clinical visit. In another example, if the comparison between the sample value and the reference value reveals that the quantity of soluble RET receptor is less than a reference value that represents the maximum quantity of soluble RET receptor that is necessary to indicate the presence of colon cancer, then such therapeutic intervention as one or more of evaluative colonoscopy, biopsy, blood counts, ultrasound, computed tomography, magnetic resonance imaging, positron emission tomography, angiography, surgery, radiation therapy, chemotherapy, and immunotherapy may be selected pursuant to developing an appropriate therapy regimen.

Because patient prognosis can vary over time, it may be appropriate to perform at least one evaluation of a subject at each of multiple time points. Thus, the present methods can further comprise obtaining a plurality of sample values in a biological fluid of the subject over time. For example, one or more sample values may be obtained at timepoint t=0, one or more additional sample values may be obtained at timepoint t=1, and a subsequent sample value or set of sample values may be obtained at timepoint t=2, wherein the amount of time between t=0 and t=1 may be one or more days, weeks, months, or years, and the amount of time between t=1 and t=2 may independently be one or more days, weeks, months, or years. Sample values may be obtained at a plurality of time points that are equally spaced, or at a plurality of time points that are not equally temporally spaced. Some disease states may be associated with rapid development, and based on such factors as the type of disease state at issue and/or the degree patient predisposition for developing a disease state, it may be appropriate to obtain sample values as often as every few months, every few weeks, or every few days.

A comparison may be made between a reference value and the sample value or set of values that is obtained at each timepoint, such that a series of comparisons are performed over time. Alternately, when a plurality of sample values in a biological fluid of the subject over time are measured, the present methods can further comprise obtaining an average of said plurality of sample values, and comparing said average with a reference value, wherein the reference value may correspond to the minimum quantity of soluble RET receptor that is necessary to indicate the presence of a disease state, or wherein the reference value may correspond to the maximum quantity of soluble RET receptor that is necessary to indicate the presence of a disease state, i.e., wherein a sample value that is below such reference value is indicative of the presence of a disease state.

Also provided are methods for detecting or aiding the detection of a disease state in a patient comprising determining the quantity of soluble RET receptor in a biological fluid of the patient, wherein an elevated quantity of soluble RET receptor compared to a threshold value indicates the presence of the disease state in the patient. In other embodiments, a decreased quantity of soluble RET receptor compared to the threshold value indicates the presence of a disease state in the patient. In such embodiments, the disease state may be, for example, colon cancer, renal cell carcinoma, lung cancer, prostate cancer, breast cancer, or pathogenic cell growth, cell migration, or angiogenesis. The threshold value may be a single numerical value expressed in terms of, e.g., concentration (mass percentage, mass volume percentage, mass volume ratio, “parts-per” notation, molarity, and the like), with or without a standard deviation, or the threshold value may comprise a range of numerical values expressed in terms of, for example, a range of concentrations, with or without standard deviations. Because it may be difficult, if not medically inappropriate, to attempt to define a single value corresponding to a quantity of soluble RET receptor that represents a “tipping point” above or below which (depending on the disease state) the disease state can definitively be said to be present, a threshold value that corresponds to a range of values is preferred.

The methods for detecting or aiding in the detection of a disease state in a patient may further comprise determining a plurality of values corresponding to the quantity of soluble RET receptor in a biological fluid of said patient over time. The technique of determining a plurality of values over time may be practiced in accordance with previously described methods. It may be especially desirable to detect certain, aspects of the development and progression of a disease state over time that are particularly indicative of carcinogenesis. In one embodiment of the present invention, the plurality of values corresponding to the quantity of soluble RET receptor in a biological fluid of said patient over time can permit an evaluation of carcinogenic progression over time. The RET receptor tyrosine kinase is a widely-recognized oncogene, and measurement of soluble RET receptor over time provides valuable insights into the existence and developmental state of carcinogenesis in a patient.

The instant methods for detecting or aiding in the detection of a disease state in a patient may additionally comprise selecting a therapy regimen based on the observed quantity of soluble RET receptor. As provided previously, the selection of a therapy regimen can embrace initiating a new therapy, altering an ongoing therapy, monitoring of a subject that is or is not undergoing therapeutic intervention, or taking no further action than had been undertaken prior to determining the quantity of soluble RET receptor.

In accordance with the present invention there are also provided methods for monitoring a patient undergoing a therapy regimen comprising determining a plurality of values corresponding to the quantity of soluble RET receptor in a biological fluid of said patient over time, and determining whether a change in said values over time has occurred. In some embodiments, an absence of change or an increase in said values over time indicates a therapeutic effect of said therapy regimen in said patient, and a decrease in said values over time indicates the absence of a therapeutic effect in said patient. As provided above and in Example 2, infra, it has been discovered that mean levels of soluble RET receptor in serum of patients in which colon cancer, renal cell carcinoma, later stage lung cancer, later stage prostate cancer, later stage breast cancer, and bladder cancer are known to be present is less than the mean value of soluble RET receptor in serum of subjects in which these disease states are known to be absent. It has also been discovered that the mean value of soluble RET receptor in plasma of subjects in which early stage breast cancer is known to be present is greater than the mean value of soluble RET receptor in plasma of female subjects in which these disease states are known to be absent. Additionally, it has been discovered that there exists a downward trend in circulating RET receptor levels as cancer stage increases, i.e., over time, with respect to at least lung cancer, prostate cancer, and breast cancer. Thus, a decreased level soluble RET receptor over time can be strongly indicative of the development or proliferation of a disease state.

In other embodiments, an absence of change or a decrease in said values over time indicates a therapeutic effect of said therapy regimen in said patient, and an increase in said values over time indicates the absence of a therapeutic effect of said therapy regimen in said patient. As provided above and in Example 2, infra, it has been discovered that there exists a upward trend in circulating RET receptor levels as cancer stage increases, i.e., over time, with respect to at least colon cancer. Thus, increasing levels of soluble RET receptor over time can be strongly indicative of the development or proliferation of a disease state.

An array of therapies may be available for a patient in which a disease state is known or suspected to be present, but a clinician is typically challenged to select and maintain therapeutic intervention that is suitably tailored to the needs of the patient. Especially in the case of anti-cancer therapies, treatment often involves harsh intervention, e.g., radiation therapy, chemotherapy, immunotherapy, surgery, adjuvant treatment, and neoadjuvant treatment, and it is especially critical to recommend and maintain only those therapies that are fully or partially efficacious. Thus, once a therapy regimen has commenced with respect to a patient, every effort should be made to determine the absence or presence of a therapeutic effect in such patient, and to that end the parameter of the quantity of soluble RET receptor in a biological fluid of the patient over the course of the treatment, i.e., over time, can reveal whether the therapy should be maintained or discontinued. For example, if over the course of three months of treatment, the quantity of soluble RET receptor in a biological fluid of said patient as measured twice per month during the three month treatment period is observed to increase over time, a positive therapeutic effect may be attributed to such treatment. If the quantity of soluble RET receptor remains the same over time, such data may also be indicative of a therapeutic effect, as, with respect to certain disease states, unchecked pathological progression is expected to give rise to lower expression levels of soluble RET receptor. An increase over time in the values corresponding to the quantity of soluble RET receptor may indicate that the chosen therapeutic route is at least partially efficacious, although a rate of decrease over time that is less rapid than the rate of decrease over time that was observed prior to the commencement of therapy may indicate that such therapy is partially efficacious. Thus, the instant methods may further comprise comparing the plurality of values corresponding to the quantity of soluble RET receptor over time to a pre-therapy reference, wherein the pre-therapy reference corresponds to the quantity of soluble RET receptor that was measured over time prior to the initiation of the therapy regimen. Preferably, the period of time over which the pre-therapy reference was obtained is substantially the same as the period of time over which the plurality of values in accordance with the present methods are determined. The therapy regimen of which an evaluation is made may comprise any affirmative therapy, such as antiangiogenic therapy, radiation therapy, chemotherapy, immunotherapy, surgery, adjuvant treatment, and neoadjuvant treatment, or may comprise a combination of therapeutic approaches.

Based on the plurality of values corresponding to the quantity of soluble RET receptor in the biological fluid of the patient over time, the instant methods of monitoring a patient undergoing a therapy regimen may further comprise altering the therapy regimen. Altering the therapy regimen may comprise changing the parameters of the existing therapy regimen, discontinuing the therapy regimen, incorporating one or more additional therapies, or initiating a new, different therapy. Thus, if, based on the determination of the plurality of values over time, it is deduced that an ongoing therapy regimen is not efficacious, such therapy can be discontinued and possibly replaced with a more aggressive treatment, such as chemotherapy or surgery.

Also provided are methods for monitoring a patient in which a disease state is known to be present comprising determining the quantity of soluble RET receptor in a biological fluid of said patient, thereby obtaining a sample value, and comparing said sample value with a reference value. The reference value may correspond to the quantity of soluble RET receptor that is known to be present in a biological fluid of a patient at a known stage of the disease state. Thus, for example, the reference value may correspond to the quantity of RET receptor that is known to be present in a biological fluid of a patient having stage 2 colon cancer. In other embodiments, the reference value may correspond to the quantity of soluble RET receptor that is known to be present in a biological fluid of said patient at an earlier time point. For example, where the sample value corresponds to the quantity of soluble RET receptor in a biological fluid of the patient at time t=1, the reference value may correspond to the quantity of soluble RET receptor in a different biological fluid sample of the patient, as obtained at time t=0, wherein t=1 corresponds to a time point that may be one or more hours, days, weeks, months, or years later than time t=0.

The instant methods for monitoring a patient in which a disease state is known to be present may further comprise determining a plurality of values corresponding to the quantity of soluble RET receptor in a biological fluid of the patient over time. The technique of determining a plurality of values “over time” may be practiced in accordance with previously described methods. When a plurality of values over time are determined, the instant methods may additionally comprise conducting a comparison among the plurality of values, thereby determining the absence or presence of one or more trends in the quantity of soluble RET receptor in the biological fluid of the patient over time. Thus, for example, the comparison among the plurality of values can be used to determine whether there exists a fully or substantially upwards trend among the plurality of values over time, a fully or substantially downwards trend among the plurality of values over time, any combination of fully or substantially upwards and fully or substantially downwards trends, or no discernable trend among the plurality of values over time.

In accordance with any of the methods of the present invention, the additional step of measuring the quantity of one or more other oncogene protein products may be performed. That is, the instant methods may further comprise measuring the quantity of at least one marker other than soluble RET receptor. For example the instant methods may further comprise measurement of the gene product of one or more of EGfr, Her2/neu, Ras, and other tyrosine kinases. The measured quantity of additional marker(s) may then respectively be compared to a known reference value for such marker, thereby providing an additional data parameter that can be used for any purpose disclosed herein with respect to soluble RET receptor. For example, with respect to the disclosed methods for monitoring a patient undergoing a therapy regimen, the methods may further comprise determining a plurality of values corresponding to the quantity of each of one or more additional markers in the biological fluid over time, thereby obtaining a value for each of said one or more additional markers for every timepoint at which a value corresponding to the quantity of soluble RET receptor is determined. Thus, if values corresponding to the quantity of soluble RET receptor are obtained at each of time t=1 and time t=2, then a value corresponding to the quantity of each of one or more additional markers in said biological fluid may also be obtained at each of time t=1 and time t=2.

The present invention also provides a purified antibody that specifically binds to a soluble form of RET receptor. The purified antibody may be monoclonal or polyclonal, the former being preferred, in one embodiment, the antibody binds to a single site on the portion of the soluble form of RET receptor that corresponds to the extracellular domain of the membrane-bound form of RET receptor. The extracellular domain of the membrane-bound form of RET receptor is found at amino acids 29 to 635 of the RET receptor tyrosine kinase, the full amino acid sequence of which is reproduced below.

```
(SEQ ID NO: 1)
                10  20  30  40  50  60
MAKATSGAAG LRLLLLLLLP LLGKVALGLY FSRDAYWEKL YVDQAAGTPL YVVHALRDAP
                70  8O  90  100  110  120
EEVPSFRLGQ HLYGTYRTRL HENNWICIQE DTGLLYLNRS LDHSSWEKLS VRNRGFPLLT
              130  140  150  160  170  180
VYLKVELSPT SLREGECQWP GCARVYFSFF NTSFPACSSL KPRELCFPET RPSFRIRENR
              190  200  210  220  230  240
PPGTFHQFRL LPVQFLCPNI SVAYRLLEGE GLPERCAPDS LEVSTRWALD REQREKYELV
              250  260  270  280  290  300
AVCTVEAGAR EEVVMVPFPV TVYDEDDSAP TFPAGVDTAS AVVEFKRKED TVVAILRVFD
              310  320  330  340  350  360
ADVVPASGEL VRRYTSTLLP GDTWAQQTFR VEHWPNETSV QANGSEVRAT VHDYRLVLNR
              370  380  390  400  410  420
NLSISENRTM QLAVLVNDSD FQGPGAGVLL LHFNVSVLPV SLHLPSTYSL SVSRRARRFA
              430  440  450  460  470  480
QIGKVCVENC QAFSGINVQY KLHSSGANCS TLGVVTSAED TSGILFVNDT KALRRPKCAE
              490  500  510  520  530  540
LHYMVVATDQ QTSRQAQAQL LVTVEGSYVA EEAGCPLSCA VSKRRLECEE CGGLGSPTGR
              550  560  570  580  390  600
CEWRQGDGKG ITRNFSTCSP STKTCPDGHC DVVETQDINI CPQDCLRGSI VGGHEPGEPR
              610  620  630  640  650  660
GIKAGYGTCN CFPEEEKCFC EPEDIQDPLC DELCRTVIAA AVLFSFIVSV LLSAFCIHCY
              670  680  690  700  710  720
HKFAEKPPIS SAEMTERRPA QAFPVSYSSS GARRPSLDSM ENQVSVDAFK ILEDPKWEFP
              730  740  750  760  770  780
RKNLVLGKTL GEGEEGKVVK ATAFHLKGRA GYTTVAVKML KENASPSELR DLLSEFNVLK
              790  800  810  820  830  840
QVNHPHVIKL YGACSQDGPL LLIVEYAKYG SLRGFLRESR KVGPGYLGSG GSRNSSSLDH
              850  860  870  880  890  900
PDERALTMGD LISFAWQISQ GMQYLAEMKL VHRDLAARNI LVAEGRKMKI SDFGLSRDVY
              910  920  930  940  950  960
EEDSYVKRSQ GRIPVKWMAI ESLFDHIYTT QSDVWSFGVL LWEIVTLGGN PYPGIPPERL
              970  980  990  1000  1010  1020
FNLLKTGHRM ERPDNCSEEM YRLMLQCWKQ EPDKRPVFAD ISKDLEKMMV KRRDYLDLAA
            1030  1040  1050  1060  1070  1080
STPSDSLIYD DGLSEEETPL VDCNNAPLPR ALPSTWIENK LYGMSDPNWP GESPVPLTRA
            1090  1100  1110
DGTNTGFPRY PNDSVYANWM LSPSAAKLMD TFDS
```

In other embodiments, the antibody binds to multiple sites on the portion of the soluble form of RET receptor that corresponds to the extracellular domain of the membrane-bound form of RET receptor. The instant purified antibodies can be produced in accordance with established protocols or using variations thereon. The purified antibodies that are specific to the soluble RET receptor may be used in a method, e.g., a therapeutic method, comprising contacting a subject with a purified antibody specific to the soluble RET receptor. Antibody-mediated targeting of the RET receptor has been proposed as a method of treatment of RET-mediated pathologies. See, e.g., Santoro M, et al., Endocrinology. 2004 December; 145(12):5448-51 Review.

Also provided are kits for detecting a soluble form of RET receptor in a biological fluid comprising, for use as a detector reagent, a purified antibody that specifically binds to a soluble form of RET receptor, and instructions for use comprising a standard curve for interpolating the quantity of the soluble form of RET receptor in the biological fluid. A standard curve may be prepared in accordance with established protocols or as provided in Example 2, infra, or as a variant thereof. The purified antibody may be directly conjugated to a signal moiety, such as inter alia, biotin, a fluorophore, a radioactive molecule, a dye. In other embodiments, the kit may further comprise a secondary antibody for binding the purified antibody, wherein the secondary antibody is conjugated to a signal moiety. The purified antibody, and where present, the secondary antibody, may be provided in premeasured aliquots. The instant kits may additionally comprise one or more of a blocking reagent, dilution reagent, buffering reagent, labeling reagent, and wash reagent, any of which may be provided in one or more premeasured aliquots.

### EXAMPLES

### Example 1

### Human Soluble RETr ELISA Assay

An assay was constructed using a goat polyclonal antibody that binds the extracellular domain of the membrane-bound form of the RET receptor. This reagent was used as a “capture” reagent in the preparation of an ELISA sandwich assay, while the detector reagent was a goat polyclonal antibody labeled with biotin. The streptavidin conjugate was linked, to horseradish peroxidase, while the substrate for colorimetric quantitation was TM Blue.

Nunc plates (96 well) were coated with 1 μg/mL anti-human soluble-RET Goat Polyclonal antibody (R&D Systems, Minneapolis, Minn.; Cat. No. AF1485) and blocked. Human serum and plasma samples were diluted in a sample diluent with goat IgG as a blocker. The standard was an insect cell-derived form of the human sRET receptor extracellular domain (R&D Systems, Minneapolis, Minn.; Cat. No. 1168-CR-050/CF). Standards and samples were incubated on, the plates for 1 hr at 37° C. The detector antibody was a biotinylated anti-human soluble-RET Goat Polyclonal antibody (R&D Systems, Minneapolis, Minn.; Cat. No. BAF1485). The plates were washed six times in phosphate wash and the detector antibody was added to the plates for 1 hr at 37° C. Plates were washed again six times and a streptavidin-HRP conjugate was added to the plates for 1 hr at room temperature. A final wash was performed as before and a TMB substrate was added to the plates for color development. The reaction was stopped with 2.5N sulfuric acid and plates were read at an absorbance of 450 nm.

### Example 2

### Sample Analysis

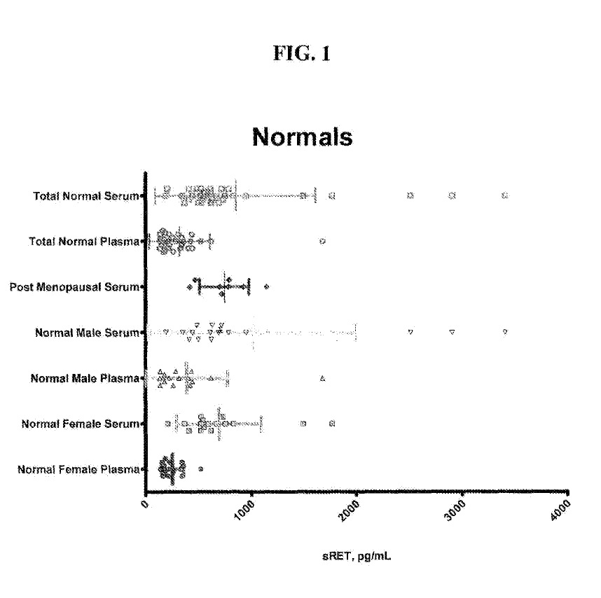

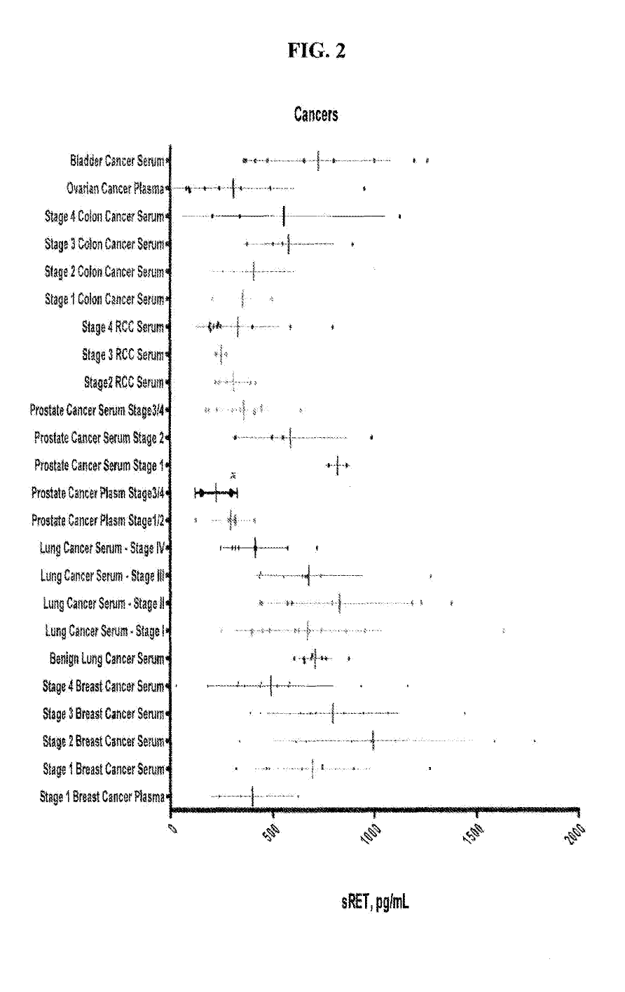

A series of human serum and human plasma samples were tested using the prototype ELISA assay described in Example 1 for the presence of circulating RET receptor. Samples were obtained from the Siemens Medical Solutions Diagnostics in-house sample bank, and were originally procured from normal human subjects and subjects in which cancer was known to be present, i.e., by type of cancer, and stage of cancer. A standard curve was constructed using known amounts of recombinant RET receptor extracellular domain protein. The values obtained from the serum and plasma samples were compared to the standard curve in order to interpolate the quantity of circulating RET receptor in the samples. Data are provided in Tables 1-9, below. FIG. 1 depicts data from measurements of soluble RET receptor in normal serum and plasma samples, whereas FIG. 2 depicts data from measurements of soluble RET receptor in samples from patients in which specific disease states are known to be present.

TABLE 1
Normal Human Samples (soluble RET, pg/mL)

| Normal | Normal | Normal | Normal | Post | | |
|---|---|---|---|---|---|---|
| Female | Female | Male | Male | Menopausal | Total Normal | Total Normal |
| Plasma | Serum | Plasma | Serum | Serum | Plasma | Serum |
| 138.802 | 522.668 | 134.957 | 947.978 | 415.540 | 138.802 | 365.805 |
| 164.162 | 665.120 | 1676.582 | 2904.083 | 696.777 | 164.162 | 537.716 |
| 223.794 | 550.959 | 178.013 | 495.006 | 1143.478 | 223.794 | 591.794 |
| 253.674 | 406.675 | 220.987 | 617.074 | 717.861 | 253.674 | 831.012 |
| 154.219 | 750.499 | 431.552 | 409.694 | 465.260 | 154.219 | 518.971 |
| 354.614 | 521.385 | 259.338 | 2512.673 | 786.177 | 354.614 | 1493.143 |
| 339.532 | 725.447 | 137.154 | 713.634 | 923.962 | 339.532 | 615.703 |
| 212.578 | 207.813 | 310.122 | 3403.131 | 780.433 | 212.578 | 1770.314 |
| 185.594 | 365.805 | 421.877 | 440.920 | | 134.957 | 947.978 |
| 327.314 | 537.716 | 614.327 | 627.320 | | 1676.582 | 2904.083 |
| 339.861 | 591.794 | 136.424 | 191.760 | | 178.013 | 495.006 |
| 516.252 | 831.012 | 439.649 | 782.256 | | 220.987 | 617.074 |
| 209.049 | 518.971 | 402.878 | 351.169 | | 431.552 | 409.694 |
| 173.275 | 1493.143 | 176.969 | 484.247 | | 259.338 | 2512.673 |
| 181.896 | 615.703 | 282.302 | 687.362 | | 137.154 | 713.634 |
| | 1770.314 | | 610.433 | | 310.122 | 3403.131 |
| | | | | | 421.877 | 522.668 |
| | | | | | 614.327 | 665.120 |
| | | | | | 136.424 | 550.959 |
| | | | | | 439.649 | 406.675 |
| | | | | | 402.878 | 750.499 |
| | | | | | 176.969 | 521.385 |
| | | | | | 282.302 | 725.447 |
| | | | | | 185.594 | 207.813 |
| | | | | | 327.314 | 440.920 |
| | | | | | 339.861 | 627.320 |
| | | | | | 516.252 | 191.760 |
| | | | | | 209.049 | 782.256 |
| | | | | | 173.275 | 351.169 |
| | | | | | 181.896 | 484.247 |
| | | | | | | 687.362 |
| | | | | | | 610.433 |

TABLE 2
Normal Human Samples (soluble RET, pg/mL)

| | Normal | Normal | Normal | Normal |
|---|---|---|---|---|
| | Male | Female | Male | Female |
| | Serum | Serum | Plasma | Plasma |
| Number of | 16 | 16 | 15 | 15 |
| values | | | | |
| Range | 192-3403 | 208-1770 | 135-1677 | 139-516 |
| Mean | 1011 | 692 | 388 | 252 |
| Std. Deviation | 987 | 400 | 383 | 104 |
| Mean + 2*SD | 2022 | 1384 | 776 | 504 |
| Lower 95% CI of | 485 | 479 | 176 | 194 |
| mean | | | | |
| Upper 95% CI of | 1537 | 905 | 601 | 309 |
| mean | | | | |

| | Post | Total | Total |
|---|---|---|---|
| | Menopausal | Normal | Normal |
| | Serum | Serum | Plasma |
| Number of | 8 | 32 | 30 |
| values | | | |
| Range | 416-1143 | 192-3403 | 135-1677 |
| Mean | 741 | 852 | 320 |
| Std. Deviation | 234 | 758 | 285 |
| Mean + 2*SD | 1482 | 1704 | 640 |
| Lower 95% CI of | 545 | 578 | 214 |
| mean | | | |
| Upper 95% CI of | 937 | 1125 | 426 |
| mean | | | |

TABLE 3
Breast Cancer Samples (soluble RET, pg/mL)

| | Stage 1 | Stage 1 | Stage 2 | Stage 3 | Stage 4 |
|---|---|---|---|---|---|
| | Breast | Breast | Breast | Breast | Breast |
| | Cancer | Cancer | Cancer | Cancer | Cancer |
| | Plasma | Serum | Serum | Serum | Serum |
| | 340.215 | 744.035 | 664.686 | 390.270 | 444.247 |
| | 235.116 | 467.895 | 335.754 | 684.884 | 578.880 |
| | 623.249 | 744.035 | 612.963 | 1442.340 | 182.039 |
| | | 481.757 | 1104.528 | 711.522 | 361.315 |
| | | 319.227 | 1781.313 | 637.699 | 583.633 |
| | | 1270.812 | 888.254 | 849.987 | 331.298 |
| | | 643.219 | 1585.492 | 947.225 | 328.118 |
| | | 898.630 | 984.328 | 1067.609 | 29.714 |
| | | | | 438.087 | 936.416 |
| | | | | | 438.087 |
| | | | | | 522.294 |
| | | | | | 1163.519 |
| Mean | 399.527 | 696.201 | 994.665 | 796.625 | 491.630 |
| % Above 95% | 33.33% | 12.50% | 50.00% | 33.33% | 16.67% |
| % Above | 0% | 0% | 25.00% | 0% | 0% |
| Mean + 2 * | | | | | |
| SD | | | | | |
| % Below 95% | 0% | 37.50% | 12.50% | 22.22% | 58.33% |

TABLE 4
Ovarian Cancer Samples (soluble RET, pg/mL)

| | Ovarian Cancer Plasma |
|---|---|
| | 489.038 |
| | 168.571 |
| | 91.063 |
| | 77.286 |
| | 344.043 |
| | 951.746 |
| | 95.265 |
| | 237.602 |
| Mean | 306.827 |
| % Above 95% | 25.00% |
| % Above | 12.50% |
| Mean + 2*SD | |
| % Below 95% | 50.00% |

TABLE 5
Bladder Cancer Samples (soluble RET, pg/mL)

| | Bladder Cancer Serum |
|---|---|
| | 413.590 |
| | 1258.337 |
| | 800.577 |
| | 1194.274 |
| | 473.830 |
| | 367.731 |
| | 999.592 |
| | 358.111 |
| | 654.364 |
| Mean | 724.490 |
| % Above 95% | 22.22% |
| % Above | 0% |
| Mean + 2*SD | |
| % Below 95% | 44.44% |

TABLE 6
Renal Cell Carcinoma Samples (soluble RET, pg/mL)

| | Stage 2 | Stage 3 | Stage 4 |
|---|---|---|---|
| | RCC Serum | RCC Serum | RCC Serum |
| | 393.177 | 273.535 | 198.046 |
| | 276.381 | 223.232 | 234.479 |
| | 222.109 | | 401.409 |
| | 416.745 | | 588.637 |
| | 238.987 | | 190.243 |
| | | | 797.646 |
| | | | 246.324 |
| | | | 228.852 |
| | | | 212.018 |
| | | | 195.257 |
| Mean | 309.480 | 248.384 | 329.291 |
| % Above 95% | 0% | 0% | 0% |
| % Above | 0% | 0% | 0% |
| Mean + 2*SD | | | |
| % Below 95% | 100% | 100% | 80.00% |

TABLE 7
Colon Cancer Samples (soluble RET, pg/mL)

| | Stage 1 | Stage 2 | Stage 3 | Stage 4 |
|---|---|---|---|---|
| | Colon | Colon | Colon | Colon |
| | Cancer | Cancer | Cancer | Cancer |
| | Serum | Serum | Serum | Serum |
| | 206.982 | 556.539 | 373.261 | 338.374 |
| | 498.638 | 255.939 | 499.847 | 1124.461 |
| | | | 546.721 | 203.629 |
| | | | 892.658 | |
| Mean | 352.810 | 406.239 | 578.122 | 555.488 |
| % Above 95% | 0% | 0% | 0% | 0% |
| % Above | 0% | 0% | 0% | 0% |
| Mean + 2*SD | | | | |
| % Below 95% | 100% | 100% | 75.00% | 100% |

TABLE 8
Prostate Cancer Samples (soluble RET, pg/mL)

| | | | Prostate | Prostate | Prostate |
|---|---|---|---|---|---|
| | Prostate | Prostate | Cancer | Cancer | Cancer |
| | Cancer | Cancer | Serum | Plasma | Plasma |
| | Serum | Serum | Stages 3 & 4 | Stages 1 & 2 | Stages 3 & 4 |
| | Stage 1 | Stage 2 | (grouped) | (grouped) | (grouped) |
| | 864.894 | 498.314 | 339.86 | 313.535 | 151.145 |
| | 776.954 | 318.544 | 639.032 | 318.543 | 296.03 |
| | | 552.248 | 174.687 | 124.176 | |
| | | 989.152 | 445.557 | 286.044 | |
| | | | 304.033 | 316.039 | |
| | | | 226.429 | 411.739 | |
| | | | 445.557 | | |
| | | | 410.992 | | |
| | | | 187.562 | | |
| | | | 410.343 | | |
| Mean | 820.924 | 589.565 | 358.405 | 295.013 | 223.588 |
| % Above | 0% | 0% | 0% | 0% | 0% |
| 95% | | | | | |
| % Above | 0% | 0% | 0% | 0% | 0% |
| Mean + | | | | | |
| 2 * SD | | | | | |
| % Below | 0% | 25.00% | 90.00% | 16.67% | 50.00% |
| 95% | | | | | |

TABLE 9
Lung Cancer Samples (soluble RET, pg/mL)

| | Benign | Lung | Lung | Lung | Lung |
|---|---|---|---|---|---|
| | Lung | Cancer | Cancer | Cancer | Cancer |
| | Cancer | Serum - | Serum - | Serum - | Serum - |
| | Serum | Stage 1 | State 2 | Stage 3 | Stage 4 |
| | 742.579 | 449.822 | 437.108 | 671.656 | 717.553 |
| | 695.227 | 1631.373 | 447.277 | 661.202 | 332.331 |
| | 875.62 | 397.819 | 792.871 | 553.536 | 409.206 |
| | 688.672 | 627.32 | 1228.42 | 440.92 | 301.028 |
| | 605.244 | 682.123 | 1376.026 | 434.567 | 243.734 |
| | 654.675 | 862.214 | 1186.381 | 1276.352 | 572.888 |
| | 657.285 | 953.833 | 593.581 | 737.304 | 316.039 |
| | 761.07 | 609.135 | 831.468 | 645.546 | 418.074 |
| | | 484.157 | 572.551 | | |
| | | 397.879 | | | |
| | | 250.846 | | | |
| | | 738.821 | | | |
| Mean | 710.047 | 673.779 | 829.520 | 677.635 | 413.857 |
| % Above | 0% | 8.33% | 33.33% | 12.50% | 0% |
| 95% | | | | | |
| % Above | 0% | 8.33% | 0% | 0% | 0% |
| Mean + 2 * SD | | | | | |
| % Below | 0% | 41.67% | 33.33% | 37.50% | 87.50% |
| 95% | | | | | |

Results demonstrated that the mean value of soluble RET receptor in normal human male plasma was lower than that of normal human serum. Normal male donors had higher levels of soluble RET receptor than that of normal female donors. Post-menopausal serum levels of soluble RET receptor matched those of normal female serum. The levels of soluble RET receptor in samples from patients having specific cancers were compared with the best-matched normal (e.g., levels of soluble RET receptor in samples from patients having breast cancer was compared to levels of soluble RET receptor in samples from normal female samples) through a comparison of 95% cutoffs.

The reference works, patents, patent applications, and scientific literature that are referred to herein are hereby incorporated by reference in their entirety to the same extent as if each was specifically and individually indicated to be incorporated by reference. Any conflict between any reference cited herein and the specific teachings of this specification shall be resolved in favor of the latter.

What is claimed:

1. A method comprising detecting a soluble form of RET receptor in a biological fluid of a subject.

2. The method according to claim 1 wherein said biological fluid is serum, plasma, sputum, acellular cerebrospinal fluid, acellular saliva, sweat, tears, urine, or any combination thereof.

3. The method according to claim 1 further comprising determining the quantity of said soluble RET receptor, thereby obtaining a sample value.

4. The method according to claim 3 wherein said sample value is determined using an ELISA assay.

5. The method according to claim 3 further comprising comparing said sample value with a reference value.

6. The method according to claim 5, wherein said reference value corresponds to a quantity of soluble RET receptor in a biological fluid of a subject in which a disease state is known to be absent.

7. The method according to claim 6 further comprising assessing the suitability of a therapy regimen based on the comparison between said sample value and said reference value.

8. The method according to claim 7 wherein the therapy regimen is a cellular RET receptor-targeted therapy.

9. The method according to claim 6 further comprising assessing the prognosis with respect to said diseases state for the subject to which the sample value corresponds.

10. The method according to claim 9 wherein said prognosis relates to rate of progression of said disease state, outcome of such disease state, or probability of recurrence of said disease state.

11. The method according to claim 5 wherein said reference value corresponds to the minimum quantity of soluble RET receptor that is necessary to indicate the presence of a disease state.

12. The method according to claim 11, wherein said disease state is cancer.

13. The method according to claim 5, further comprising selecting a therapy regimen based on said comparison of said sample value with said reference value.

14. The method according to claim 5 wherein said reference value corresponds to the quantity of soluble RET receptor above which denotes the presence of a disease state.

15. The method according to claim 14, wherein said disease state is cancer.

16. The method according to claim 14, further comprising selecting a therapy regimen based on said comparison of said sample value with said reference value.

17. The method according to claim 3 comprising obtaining a plurality of sample values in a biological fluid of said subject over time.

18. The method according to claim 1 further comprising measuring one or more additional markers in said biological fluid.

19. A method for detecting or aiding the detection of a disease state in a patient comprising determining the quantity of soluble RET receptor in a biological fluid of said patient, wherein a quantity of soluble RET receptor that is at variance with a reference normal value indicates the presence of said disease state in said patient.

20. The method according to claim 19 wherein a decreased quantity of soluble RET receptor as compared with a reference normal value indicates the presence of said disease state in said patient.

21. The method according to claim 20 wherein said disease state is cancer.

22. The method according to claim 19 wherein an elevated quantity of soluble RET receptor as compared with a reference normal value indicates the presence of said disease state in said patient.

23. The method according to claim 22 wherein said disease state is cancer.

24. The method according to claim 19 comprising determining a plurality of values corresponding to the quantity of said soluble RET receptor in a biological fluid of said patient over time.

25. The method according to claim 19, further comprising measuring one or more additional markers in said biological fluid.

26. The method according to claim 24 further comprising evaluating cancer stage progression over time based on said plurality of values.

27. The method according to claim 19 further comprising selecting a therapy regimen based on said quantity of soluble RET receptor.

28. A method for monitoring a patient undergoing a therapy regimen comprising:
determining a plurality of values corresponding to the quantity of soluble RET receptor in a biological fluid of said patient over time; and,
determining whether a change in said values over time has occurred.

29. The method according to claim 28 wherein an absence of change or a decrease in said values over tune indicates a therapeutic effect of said therapy regimen in said patient, and wherein an increase in said values over tune indicates the absence of a therapeutic effect of said therapy regimen in said patient.

30. The method according to claim 28 wherein an absence of change or an increase in said values over time indicates a therapeutic effect of said therapy regimen in said patient, and wherein a decrease in said values over time indicates the absence of a therapeutic effect of said therapy regimen in said patient.

31. The method according to claim 28 further comprising, based on said plurality of values over time, altering said therapy regimen.

32. The method according to claim 28 further comprising determining a plurality of values corresponding to the quantity of each of one or more additional markers in said biological fluid over time, thereby obtaining a value for each of said one or more additional markers for every timepoint at which a value corresponding to the quantity of soluble RET receptor is determined.

33. A purified antibody that specifically binds to a soluble form of RET receptor.

34. A method comprising contacting a subject with an antibody according to claim 33.

35. A kit for detecting a soluble form of RET receptor in a biological fluid comprising:
an antibody according to claim 33 for use as a detector reagent; and,
instructions for use comprising a standard curve for interpolating the quantity of said soluble form of RET receptor in said biological fluid.

36. The kit according to claim 35 wherein said antibody is conjugated to a signal moiety.

37. The kit according to claim 35 further comprising a secondary antibody for binding said antibody that binds to a soluble form of RET receptor, wherein said secondary antibody is conjugated to a signal moiety.

38. A method for monitoring a patient in which a disease state is known to be present comprising determining the quantity of soluble RET receptor in a biological fluid of said patient, thereby obtaining a sample value, and comparing said sample value with a reference value.

39. The method according to claim 38 wherein said disease state is cancer.

40. The method according to claim 38 wherein said reference value corresponds to the quantity of soluble RET receptor known to be present in a biological fluid of a patient at a known stage of said disease state.

41. The method according to claim 38 wherein said reference value corresponds to the quantity of soluble RET receptor known to be present in a biological fluid of said patient at an earlier time point.

42. The method according to claim 38 further comprising determining a plurality of values corresponding to the quantity of soluble RET receptor in a biological fluid of said patient over time.

43. The method according to claim 42 further comprising conducting a comparison among said plurality of values, thereby determining the absence or presence of one or more trends in the quantity of said soluble RET receptor in said biological fluid of said patient over time.

44. The method according to claim 38, further comprising measuring one or more additional markers in said biological fluid.
